(19) United States
(12) Patent Application Publication
(10) Pub. No.: US 2008/0154194 A1
(43) Pub. Date: Jun. 26, 2008

(54) Safety syringe device with retractable needle cannula

(76) Inventor: Hung-Chi Huang, Taichung City (TW)

Correspondence Address: Hung-Chi Huang, P.O. BOX 44-2049, Taipei, 10668, omitted

(21) Appl. No.: 11/642,526

(22) Filed: Dec. 21, 2006

(51) Int. Cl. A61M 5/50

(52) U.S. Cl. 604/110

(57) ABSTRACT

A safety syringe device includes a barrel having a passage defined therethrough and an engaging space is defined in the passage which has a tapered inner periphery and is located close to an end of the barrel. A connection member is inserted into the barrel and engaged with the engaging space. A part of the connection member extends out from an opening of the end of the barrel so as to be connected with a needle hub. The connection member is a tapered member which has an oval recess. A plunger has a stopper connected to an end thereof and an insertion extends from the stopper. A maximum diameter of the insertion is 85% to 95% of an inner diameter of the barrel. The insertion is engaged with the oval recess and expands the oval recess to securely connect the insertion and the connection member.

### BACKGROUND OF THE INVENTION

(1) Field of the Invention

The present invention relates to a syringe device and the needle cannula can be pulled into the barrel after use.

(2) Description of the Prior Art

A conventional syringe device generally includes a barrel and a plunger is movably inserted into the first end of a barrel, a needle cannula is connected to a second end of the barrel. The plunger has a stopper at an end thereof and the stopper is in contact with an inner periphery of the barrel so that when pushing the plunger toward the needle cannula, the medicine in the barrel is ejected from the needle cannula. However, the inner diameter of the barrel is constant and the stopper is in contact with the inner periphery of the barrel by a limited area so that if the plunger is pushed too fast, some medicine might leak into the barrel behind the stopper.

The size of the stopper has to be carefully calculated so as to have a desired feature. If the size of the stopper is too small, there will be a gap between the stopper and the inner periphery of the barrel. On the contrary, if the size of the stopper is too large, when pulling the plunger backward, the stopper might be disengaged from the plunger.

The present invention intends to provide a safety syringe device wherein a connection member is inserted into the barrel and the needle hub is connected to the connection member. The connection member has an oval recess. The stopper includes an insertion which is engaged with the oval recess and expands the oval recess to a circular recess so that the connection member, the needle hub and the stopper are securely connected with the insertion. When the plunger is pulled backward, the needle hub is pulled into the barrel after injection.

### SUMMARY OF THE INVENTION

The present invention relates to a safety syringe device which comprises a barrel which has a finger flange at the first end of the barrel and a circular flange is connected to the second end of the barrel so as to define an opening. A passage is defined through the barrel and has an engaging space which has a tapered inner periphery which is located close to the second end of the barrel. A connection member is inserted into the barrel from the first end of the barrel and engaged with the engaging space. A first part of the connection member extends out from the opening of the barrel so as to be connected with a needle hub. The second part of the connection member is a tapered member which has an oval recess. The tapered member has a tapered outer periphery which is matched with the tapered inner periphery of the engaging space. A plunger has a stopper connected to an end thereof and a rubber ring is mounted on the stopper. An insertion extends from the stopper and shares a common axis with the rubber ring. A maximum diameter of the insertion is smaller than the diameter of the rubber ring and is 85% to 95% of an inner diameter of the barrel.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is an exploded view to show the safety syringe device of the present invention;

FIG. 1A shows the connection member of the safety syringe device of the present invention;

FIG. 2 is a cross sectional view to show that the connection member is inserted into the barrel so as to be connected with the needle hub;

FIG. 3 shows that the needle hub is connected to the connection member;

FIG. 4 shows that the insertion is moved toward the connection member when the plunger is pushed into the barrel;

FIG. 5 shows that the insertion is connected with the connection member;

FIG. 6 shows that the plunger is pulled backward to pull the needle hub into the barrel, and the plunger is broken;

FIG. 7 shows that the insertion is not yet inserted into the oval recess of the connection member, and

FIG. 8 shows that the insertion is inserted into the oval recess of the connection member and expands the oval recess to be a circular recess.

### DETAILED DESCRIPTION OF THE PREFERRED EMBODIMENT

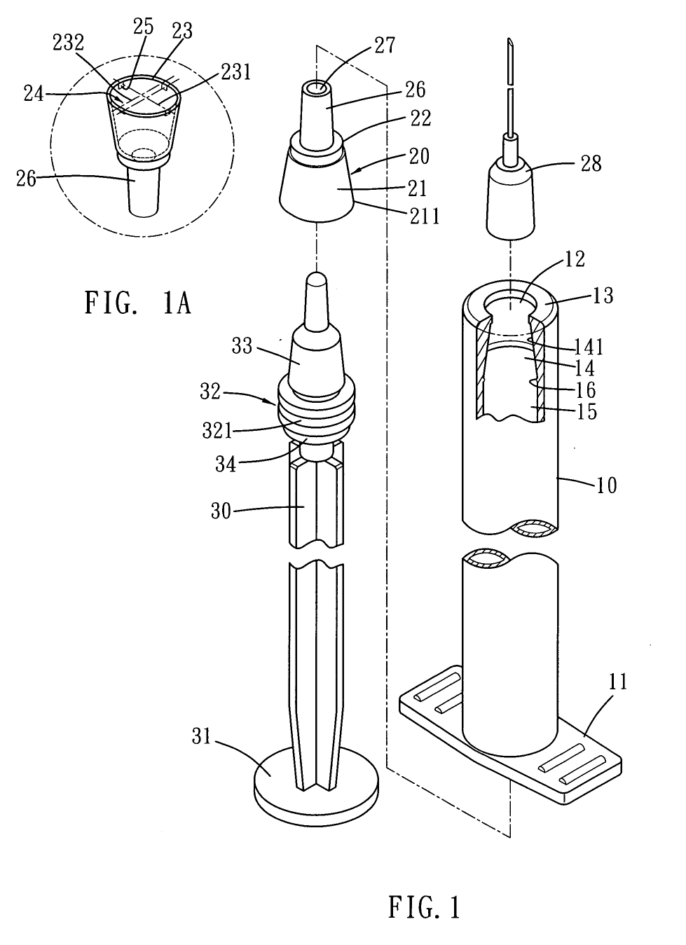

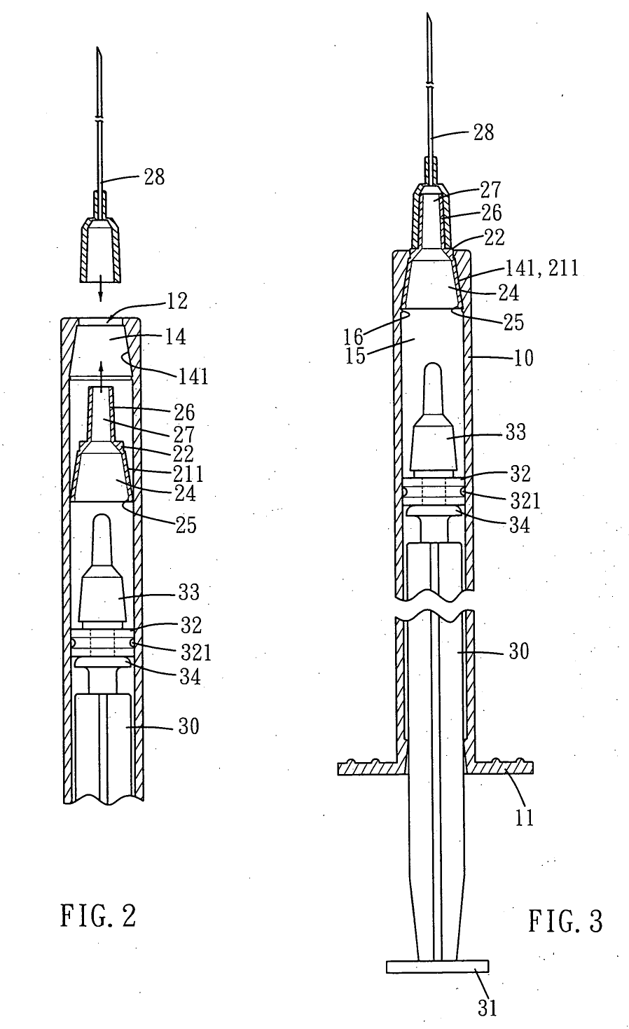

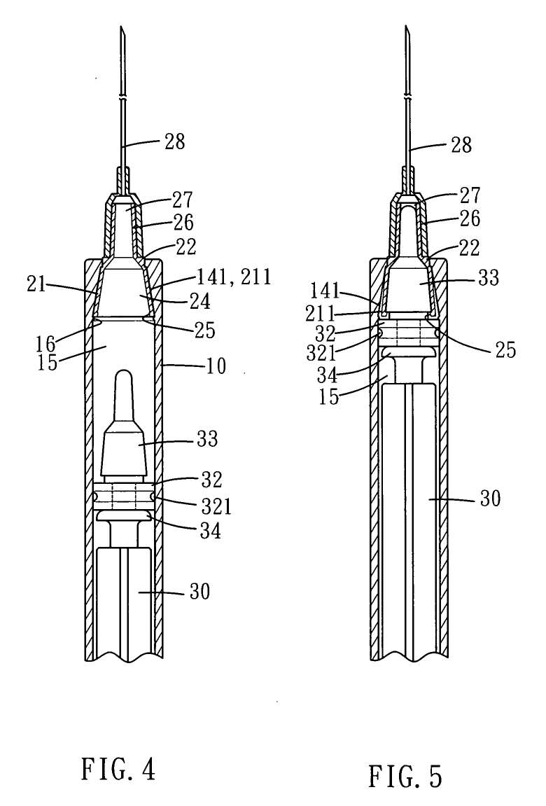

Referring to FIGS. 1 to 5, the safety syringe device of the present invention comprises a barrel 10 which has a first end and second end. A finger flange 11 is connected to the first end of the barrel 10 and a circular flange 13 is connected to the second end of the barrel 10 so as to define an opening 12. A passage 15 is defined through the barrel 10 and communicates with the first end of the barrel 10 and the opening 12 of the second end of the barrel 10. The passage 15 has an engaging space 14 which has a tapered inner periphery and is located close to the second end of the barrel 10. A stop ridge 16 extends radially inward from the inner periphery of the passage 15 and is located at a joint portion of the engaging space 14 and the passage 15.

A connection member 20 has a first part and a second part, the connection member 20 is inserted into the barrel 10 from the first-end of the barrel 10 and is engaged with the engaging space 14. The first part extends out from the opening 12 of the barrel 10 and a needle hub 28 is connected to the first part of the connection member 20. A needle cannula is connected to the needle hub 28. The second part of the connection member 20 is a tapered member 21 which has a circular shoulder 22 at a first end thereof and an oval recess 24 is defined in a second end of the tapered member 21. A plurality of hooks 25 extend from an inner periphery of the oval recess 24 of the tapered member 21. A connection tube 26 extends from the first end of the tapered member 21 so that the needle hub 28 is mounted thereto. An axial path 27 is defined axially through the connection tube 26 such that the medicine can be ejected out from the needle cannula via the axial path 27. A long axis 231 of the oval recess 24 is larger than or equal to a maximum diameter of the insertion 33, and a short axis 232 of the oval recess 24 is smaller than or equal to an maximum diameter of the insertion 33. The tapered member 21 has a tapered outer periphery 211 which is matched with the tapered inner periphery of the engaging space 14. The second end of the connection member 20 is engaged with the stop ridge 16 so that the connection member 20 is positioned in the barrel 10.

A plunger 30 has a thumb rest 31 at a first end thereof and a stopper 32 is connected to a second end of the plunger 30. A rubber ring 321 is mounted on the stopper 32 and an insertion 33 extends from the stopper 32 and shares a common axis with the rubber ring 321. A maximum diameter of the insertion 33 is smaller than the diameter of the rubber ring 321 and is 85% to 95% of an inner diameter of the barrel 10.

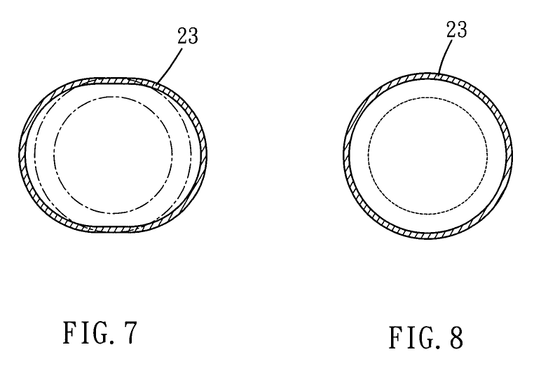

When the plunger 30 is moved into the passage 15 of the barrel 10, the medicine is ejected out from the needle cannula and the insertion 33 is inserted into the oval recess 24 of the connection member 20 when the plunger 30 is moved to its extreme position after the injection as shown in FIG. 5. As shown in FIG. 7, when the insertion 33 is not yet inserted into the oval recess 24, the oval recess 24 is maintained to be oval and the connection member 20 is positioned by the engagement between the tapered outer periphery 211 of the tapered member 21 and the tapered inner periphery of the engaging space 14, and by the stop ridge 16.

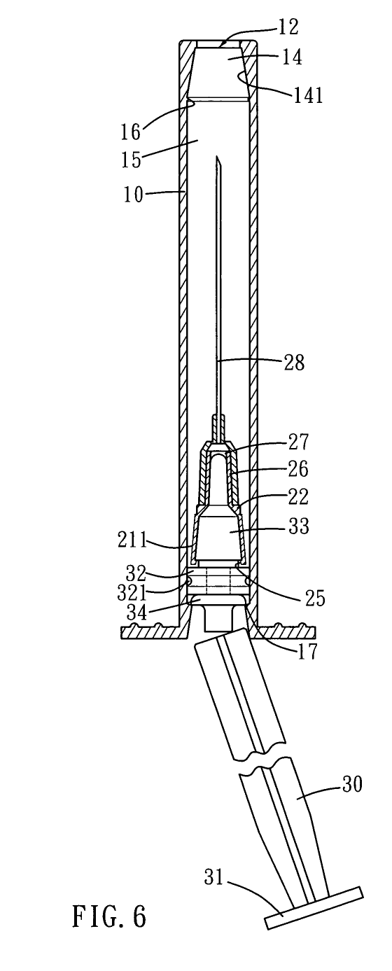

As shown in FIG. 8, when the insertion 33 is inserted into the oval recess 24 of the connection member 20, the insertion 33 expands the oval recess 24 to be a circular recess. The long axis 231 becomes shorter so as to allow the connection member 20 to pass through the space enclosed by the stop ridge 16. The hooks 25 on the inner periphery of the oval recess 24 of the tapered member 21 hook a neck portion of the insertion 33 so that when the plunger 30 is pulled backward, the connection member 20, the needle hub 28 and the stopper 32 are pulled into the barrel 10. The plunger 30 is then broken as shown in FIG. 6.

While we have shown and described the embodiment in accordance with the present invention, it should be clear to those skilled in the art that further embodiments may be made without departing from the scope of the present invention.

What is claimed is:

1. A safety syringe device comprising:
a barrel having a first end and second end, a finger flange connected to the first end of the barrel and a circular flange connected to the second end of the barrel so as to define an opening, a passage defined through the barrel and communicating with the first end of the barrel and the opening of the second end of the barrel, the passage having an engaging space which has a tapered inner periphery which is located close to the second end of the barrel;
a connection member having a first part and a second part, the connection member inserted into the barrel from the first end of the barrel and engaged with the engaging space, the first part extending out from the opening of the barrel and a needle hub connected to the first part of the connection member, the second part of the connection member being a tapered member which has an oval recess, the tapered member having a tapered outer periphery which is matched with the tapered inner periphery of the engaging space, and
a plunger having a thumb rest at a first end thereof and a stopper is connected to a second end of the plunger, a rubber ring mounted on the stopper and an insertion extending from the stopper and sharing a common axis with the rubber ring, a maximum diameter of the insertion being smaller than the diameter of the rubber ring and being 85% to 95% of an inner diameter of the barrel.

2. The device as claimed in claim 1, wherein the tapered member has a circular shoulder at a first end thereof and the oval recess is defined in a second end of the tapered member, a connection tube extends from the first end of the tapered member so that the needle hub is mounted thereto, an axial path is defined axially through the connection tube, a long axis of the oval recess being larger than or equal to an maximum diameter of the insertion, a short axis of the oval recess being smaller than or equal to an maximum diameter of the insertion.

3. The device as claimed in claim 1, wherein a stop ridge extends radially inward from the inner periphery of the passage and is located at a joint portion of the engaging space and the passage.

4. The device as claimed in claim 1, wherein a plurality of hooks extend from an inner periphery of the oval recess of the tapered member so as to hook a neck portion of the insertion.
